(19) United States
(12) Patent Application Publication
(10) Pub. No.: US 2020/0113800 A1
(43) Pub. Date: Apr. 16, 2020

(54) COSMETIC COMPOSITION

(71) Applicants: Nicole Manke, New York, NY (US); Alessandra Sozzi, New York, NY (US)

(72) Inventors: Nicole Manke, New York, NY (US); Alessandra Sozzi, New York, NY (US)

(21) Appl. No.: 16/159,175

(22) Filed: Oct. 12, 2018

(51) Int. Cl. A61K 8/49; A61Q 19/08; A61Q 19/06

(52) U.S. Cl. CPC A61K 8/492; A61K 8/9789; A61Q 19/06; A61Q 19/08

(57) ABSTRACT

Cosmetics or similar dermatological or pharmaceutical preparations containing materials, or derivatives thereof, of indole-3-carbinol for the intended use of improving the appearance of the skin

### FIELD OF INVENTION

The present arrangement relates to cosmetic or similar dermatological or pharmaceutical preparations intended for improving the appearance of the skin.

### BACKGROUND

Having a nice, attractive, or otherwise positively-held appearance is highly favored. In particular, the appearance of skin has solidified itself as an important factor in one's overall appearance.

The human skin, being the largest organ, is subject to many environmental and physical factors that can have an effect on its appearance. These effects can cause the appearance of unevenness, surface tension, blemishes, unfavorable texture and conditioning, and puffiness.

While many products have been created in attempts to improve these appearances, there is a continual search for different, more effective solutions.

### DESCRIPTION OF INVENTION

The present arrangement is directed to a topical or otherwise transdermal cosmetics or similar dermatological or pharmaceutical preparations composition containing an effective amount of indole-3-carbinol and/or its derivatives as an active ingredient, in a physiologically acceptable medium, for the purpose of improving the appearance of the skin and diminishing wrinkles, fine lines, blemishes, skin discoloration, unfavorable skin texture, cellulite, bruising and superficial spider veins.

The composition can contain at least one form of indole-3-carbinol and/or at least one form of its derivatives obtained from any material originating from a Brassica family plant cultivated in vivo or originating from in vitro culture.

Applicants note that indole-3-carbinol is a compound derived from the enzymatic hydrolysis (breakdown) of glucosinolates in plants belonging to the Brassica genus, commonly known as cruciferous vegetables.

The Brassica genus belongs to the Brassicaceae family, and as such the Brassicales order. The genus contains 20 species: Brassica alboglabra, Brassica carinata, Brassica chinensis, Brassica cretica, Brassica elongata, Brassica fruticulosa, Brassica juncea, Brassica napus, Brassica narinosa, Brassica nigra, Brassica oleracea, Brassica parachinensis, Brassica perviridis, Brassica rapa, Brassica rupestris, Brassica ruvo, Brassica septiceps, Brassica sisymbrioides, and Brassica tournefortii.

Preferentially, in the present arrangement, the material of indole-3-carbinol and/or its derivatives originates from one or more of these species.

Derivatives of indole-3-carbinol are understood to be acid condensation products, that are derived through chemical processes, frequently those in stomach acid. The acid condensation products worth mentioning in relation to the invention are 3,3′-diindolylmethane (DIM), 5,11-dihydroindolo-[3,2-b]carbazole (ICZ), and 5,6,11,12,17,18-hexahydrocyclonona[1,2-b:4,5-b′:7,8-b″]triindole. Preferentially, the composition contains the indole-3-carbinol acid condensation product 3,3′-diindolylmethane (DIM).

In one arrangement, the present invention contemplates either one of in vivo or in vitro cultivated 3,3′-diindolylmethane (DIM).

In vivo cultivation is understood as meaning any cultivation in soil in the open air or in a greenhouse, or alternatively outside the soil. In vitro culture is understood as meaning all of the techniques known to the person skilled in the art which artificially allow the obtainment of a plant or of a part of a plant.

Regarding the effective amount of 3,3′-diindolylmethane (DIM) contained in the present composition, in a preferred embodiment it can contain between 0.5% and 50% material of indole-3-carbinol and/or its derivatives. In a more preferred embodiment, the composition contains between 0.5% and 10% of the indole-3-carbinol acid condensation product 3,3′-diindolylmethane (DIM). Although, Applicants note the expected minimum effective dose is 0.5% it is possible with certain additional components and particular holding mediums, such minimum percentage may be slightly adjusted.

Regarding the medium, in one embodiment the effective amount of indole-3-carbinol/3,3′-diindolylmethane (DIM) may be included in any type of stable medium including but not limited to oil based, water based or alcohol based mediums.

In a preferred embodiment the effective amount of indole-3-carbinol/3,3′-diindolylmethane (DIM) is suspended in coconut oil.

Although no specific formulation is required apart from including an effective amount of indole-3-carbinol/3,3′-diindolylmethane (DIM), at least one specific embodiment of the present composition is formulated as followed and was applied to a test subject for efficacy testing.

In one example, a composition was prepared with a 3.24% DIM concentration in base medium formula of 58% distilled water, 12.97% petrolatum, 8.56% sea kelp extract, 7.13% cetyl alcohol, sodium polyacrylate, polsorbate 80, glyceryl stearate, dimethicone/divinyldimethicone/silsesquioxane crosspolymer mixture, 6.5% organic cold pressed pure hazelnut oil, 3.24% organic extra virgin coconut oil, and <1% parfum. In this example phenoxyenthanol as a preservative, and colorant was used. This exemplary formula was applied to one test subject twice daily (morning and late afternoon/evening) on and on-going basis for two weeks. This exemplary formula including an effective amount of DIM produced results as soon as 5 minutes after the first application with results continuing to improve upon subsequent applications. The results included but are not limited to: lessened appearance of wrinkles, fine lines, skin discoloration, unfavorable skin texture, blemishes, cellulite, bruising, and superficial spider veins. Other examples are contemplated that include similar concentrations of the effective amount of indole-3-carbinol/3,3′-diindolylmethane (DIM) within a similar medium.

In other embodiments, the composition can, but is not limited to, be additionally formulated with any one or more of the following additive ingredients, in any of their forms, in order to provide a wider range of cosmetic purposes: water, Vitamin C, Vitamin E, Vitamin D, Vitamin K, Vitamin B, Vitamin B2, Vitamin B3, Vitamin B4, Vitamin B5, Vitamin B6, Vitamin B9, Vitamin B12, Vitamin H, calcium, potassium, phosphorus, magnesium, iron, copper, zinc, kelp, algae, collagen, beta-carotene, alanine, arginine, asparagine, aspartic acid, cysteine, glutamine, glutamic acid, glycine, histidine, isoleucine, leucine, lysine, methionine, phenylalanine, piperine, proline, serine, threonine, tryptophan, tyrosine, valine, selenocysteine, pyrrolysine, glutathione, hyaluronic acid, gamma-aminobutyric acid, kaolin, caprylic/capric triglyceride, petrolatum, sunflower wax, sodium polyacrylate, glycerin, isohexadecane, cholesterol, hazelnut oil, coconut oil, shea butter, almond butter, almond oil, avocado butter, avocado oil, argan oil, acrylates C10-30 alkyl acrylate crosspolymer, sorbitan laurate, polyglyceryl-4 laurate, dilauryl citrate, cetyl alcohol, sodium polyacrylate, polsorbate 80, glyceryl stearate, dimethicone/divinyldimethicone/silsesquioxane crosspolymer, stearic acid, ceteareth-20, cetearyl alcohol, and others.

In other embodiments, the composition in its effective amount as a composition can be included in and/or take the form of any one of the group consisting of creams, lotions, milks or creams, ointments, gels, masks, mousses, scrubs, emulsions (water-in-oil and/or oil-in-water), dispersions, solutions, suspensions, cleansers, foundations, anhydrous preparations (sticks in particular lip balm, body and bath oils), shower and bath gels, porous solids, shampoo and hair care lotions, milks or creams for skin care or hair, cleansing lotions or milks, sunscreen lotions, milks or creams, artificial tanning lotions, milks or creams, pre shave, shaving or aftershave creams, foams, gels or lotions, makeup, lipstick, foam in the form of aerosol compositions also comprising a pressurized propellant, mascara or nail polish, skin essences, serums, adhesive or absorbent materials, transdermal patches, or emollient powders, lotions, milks or creams, sprays, body and bath oils, foundation basis, ointment, emulsion, colloid, compact suspension or solid, deodorant, anti-perspirant, pencil, sprayable formulation, brushable, blush, red, eyeliner, lipliner, lip gloss, face or body powder, styling gels or mousses, nail conditioning, lip balms, skin conditioners, moisturizers, lacquers, soaps, exfoliants, etc. These compositions may also be in the form of lipsticks intended either to color the lips or to prevent them from chapping, or makeup for eyes, eyes-shadows and foundations for the face. The compositions for the invention can include cosmetics, personal care products and pharmaceutical preparations.

In one embodiment the combined effective amount of indole-3-carbinol/3,3′-diindolylmethane (DIM) as suspended in the application medium is stored in sealed but not air-tight glass, plastic, porcelain, and/or acrylic containers. Stabilizers and emulsifiers included in formulation above (e.g. cetyl alcohol, sodium polyacrylate, polsorbate 80, glyceryl stearate, dimethicone/divinyldimethicone/silsesquioxane crosspolymer mixture and phenoxyethanol as a preservative) may be further included.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

What is claimed is:

1. A method of diminishing wrinkles, fine lines, blemishes, skin discoloration, unfavorable skin texture, cellulite, bruising and superficial spider veins in human skin comprising administering an effective dose of a compound indole-3-carbinol to human skin in need of diminishment in wrinkles, fine lines, blemishes, skin discoloration, unfavorable skin texture, cellulite, bruising and superficial spider veins.

2. The method as claimed in claim 1, wherein said indole-3-carbinol is selected from the group consisting of 3,3′-diindolylmethane (DIM), 5,11-dihydroindolo-[3,2-b]carbazole (ICZ), and 5,6,11,12,17,18-hexahydrocyclonona[1,2-b:4,5-b′: 7,8-b″ ]triindole.

3. The method as claimed in claim 2, wherein said indole-3-carbinol is 3,3′-diindolylmethane (DIM).

4. The method as claimed in claim 1, wherein said indole-3-carbinol is derived from an enzymatic hydrolysis of glucosinolates in plants belonging to the Brassica genus.

5. The method as claimed in claim 1, wherein said indole-3-carbinol is suspended in a medium that is any one of oil based, water based, or alcohol based.

6. The method as claimed in claim 1, wherein said method further includes the application of an additional component selected from the group consisting of water, Vitamin C, Vitamin E, Vitamin D, Vitamin K, Vitamin B1, Vitamin B2, Vitamin B3, Vitamin B4, Vitamin B5, Vitamin B6, Vitamin B9, Vitamin B12, Vitamin H, calcium, potassium, phosphorus, magnesium, iron, copper, zinc, kelp, algae, collagen, beta-carotene, alanine, arginine, asparagine, aspartic acid, cysteine, glutamine, glutamic acid, glycine, histidine, isoleucine, leucine, lysine, methionine, phenylalanine, piperine, proline, serine, threonine, tryptophan, tyrosine, valine, selenocysteine, pyrrolysine, glutathione, hyaluronic acid, gamma-aminobutyric acid, kaolin, caprylic/capric triglyceride, petrolatum, sunflower wax, sodium polyacrylate, glycerin, isohexadecane, cholesterol, hazelnut oil, coconut oil, shea butter, almond butter, almond oil, avocado butter, avocado oil, argan oil, acrylates C10-30 alkyl acrylate crosspolymer, sorbitan laurate, polyglyceryl-4 laurate, dilauryl citrate, cetyl alcohol, sodium polyacrylate, polsorbate 80, glyceryl stearate, dimethicone/divinyldimethicone/silsesquioxane crosspolymer, stearic acid, ceteareth-20, and cetearyl alcohol.

7. The method as claimed in claim 1, wherein said method further includes incorporating the effective amount of said indole-3-carbinol into a medium so as to generate a product selected from the group consisting of creams, lotions, milks or creams, ointments, gels, masks, mousses, scrubs, emulsions (water-in-oil and/or oil-in-water), dispersions, solutions, suspensions, cleansers, foundations, anhydrous preparations (sticks in particular lip balm, body and bath oils), shower and bath gels, porous solids, shampoo and hair care lotions, milks or creams for skin care or hair, cleansing lotions or milks, sunscreen lotions, milks or creams, artificial tanning lotions, milks or creams, pre shave, shaving or aftershave creams, foams, gels or lotions, makeup, lipstick, foam in the form of aerosol compositions also comprising a pressurized propellant, mascara or nail polish, skin essences, serums, adhesive or absorbent materials, transdermal patches, or emollient powders, lotions, milks or creams, sprays, body and bath oils, foundation basis, ointment, emulsion, colloid, compact suspension or solid, deodorant, anti-perspirant, pencil, sprayable formulation, brushable, blush, red, eyeliner, lipliner, lip gloss, face or body powder, styling gels or mousses, nail conditioning, lip balms, skin conditioners, moisturizers, lacquers, soaps, and exfoliants.

8. A product for diminishing wrinkles, fine lines, blemishes, skin discoloration, unfavorable skin texture, cellulite, bruising, and superficial spider veins in human skin comprising: an effective dose of a compound indole-3-carbinol for application to human skin in need of diminishment in wrinkles, fine lines, blemishes, skin discoloration, unfavorable skin texture, cellulite, bruising and superficial spider veins.

9. The product as claimed in claim 8, wherein said indole-3-carbinol is selected from the group consisting of 3,3′-diindolylmethane (DIM), 5,11-dihydroindolo-[3,2-b]carbazole (ICZ), and 5,6,11,12,17,18-hexahydrocyclonona[1,2-b:4,5-b′: 7,8-b″]triindole.

10. The product as claimed in claim 9, wherein said indole-3-carbinol is 3,3′-diindolylmethane (DIM).

11. The product as claimed in claim 8, wherein said indole-3-carbinol is derived from an enzymatic hydrolysis of glucosinolates in plants belonging to the Brassica genus.

12. The product as claimed in claim 8, wherein said indole-3-carbinol is suspended in a medium that is any one of oil based, water based, or alcohol based.

13. The product as claimed in claim 1, wherein said product further includes an additional component selected from the group consisting of water, Vitamin C, Vitamin E, Vitamin D, Vitamin K, Vitamin B1, Vitamin B2, Vitamin B3, Vitamin B4, Vitamin B5, Vitamin B6, Vitamin B9, Vitamin B12, Vitamin H, calcium, potassium, phosphorus, magnesium, iron, copper, zinc, kelp, algae, collagen, beta-carotene, alanine, arginine, asparagine, aspartic acid, cysteine, glutamine, glutamic acid, glycine, histidine, isoleucine, leucine, lysine, methionine, phenylalanine, piperine, proline, serine, threonine, tryptophan, tyrosine, valine, selenocysteine, pyrrolysine, glutathione, hyaluronic acid, gamma-aminobutyric acid, kaolin, caprylic/capric triglyceride, petrolatum, sunflower wax, sodium polyacrylate, glycerin, isohexadecane, cholesterol, hazelnut oil, coconut oil, shea butter, almond butter, almond oil, avocado butter, avocado oil, argan oil, acrylates C10-30 alkyl acrylate crosspolymer, sorbitan laurate, polyglyceryl-4 laurate, dilauryl citrate, cetyl alcohol, sodium polyacrylate, polsorbate 80, glyceryl stearate, dimethicone/divinyldimethicone/silsesquioxane crosspolymer, stearic acid, ceteareth-20, and cetearyl alcohol.

14. The product as claimed in claim 1, wherein said product incorporating the effective amount of said indole-3-carbinol into a medium is a product selected from the group consisting of creams, lotions, milks or creams, ointments, gels, masks, mousses, scrubs, emulsions (water-in-oil and/or oil-in-water), dispersions, solutions, suspensions, cleansers, foundations, anhydrous preparations (sticks in particular lip balm, body and bath oils), shower and bath gels, porous solids, shampoo and hair care lotions, milks or creams for skin care or hair, cleansing lotions or milks, sunscreen lotions, milks or creams, artificial tanning lotions, milks or creams, pre shave, shaving or aftershave creams, foams, gels or lotions, makeup, lipstick, foam in the form of aerosol compositions also comprising a pressurized propellant, mascara or nail polish, skin essences, serums, adhesive or absorbent materials, transdermal patches, or emollient powders, lotions, milks or creams, sprays, body and bath oils, foundation basis, ointment, emulsion, colloid, compact suspension or solid, deodorant, anti-perspirant, pencil, sprayable formulation, brushable, blush, red, eyeliner, lipliner, lip gloss, face or body powder, styling gels or mousses, nail conditioning, lip balms, skin conditioners, moisturizers, lacquers, soaps, and exfoliants.
